(19) United States
(12) Patent Application Publication
(10) Pub. No.: US 2022/0395475 A1
(43) Pub. Date: Dec. 15, 2022

(54) AFRAMOMUM MELEGUETA COMPOSITION AND METHOD FOR HEALTH MAINTENANCE

(71) Applicant: Kodimule Shyam PRASAD, Bangalore (IN)

(72) Inventor: Kodimule Shyam PRASAD, Bangalore (IN)

(21) Appl. No.: 17/726,519

(22) Filed: Apr. 21, 2022

Related U.S. Application Data
(60) Provisional application No. 63/178,514, filed on Apr. 22, 2021.

(51) Int. Cl. A61K 31/12; A61K 36/906; A61K 9/48; A61K 9/00; A61P 3/04

(52) U.S. Cl. CPC A61K 31/12; A61K 36/906; A61K 9/48; A61K 9/0053; A61P 3/04

(57) ABSTRACT

The invention provides a composition comprising 6-paradol as an effective ingredient in promoting thermogenesis. The composition may further contain one or more of 6-shogaol and 6-gingerol as active ingredients. The composition can be an extract of Aframomum melegueta seeds that is standardized for 6-paradol, 6-shogaol, and/or 6-gingerol. The composition can be administered in methods for promoting or maintaining a healthy body weight, as well as for promoting a healthy liver and supporting liver function.

### CROSS-REFERENCE TO RELATED APPLICATIONS

This application claims priority to provisional application No. 63/178,514 filed Apr. 22, 2021, the entire contents of which are incorporated herein by reference for all purposes.

### FIELD OF INVENTION

The invention generally relates to herbal preparations for supporting and maintaining health. More particularly, the invention relates to a composition derived from Aframomum melegueta and methods for its use in promoting health.

### BACKGROUND OF THE INVENTION

Obesity and type 2 diabetes present a serious health concern globally. An imbalance in energy intake and expenditure results in weight gain and obesity. Excess weight is stored in the form of lipids in white adipose tissue (WAT). In addition to these fat-storing cells, brown adipocytes also exist which can transform energy into heat, a process known as thermogenesis. Activation of thermogenesis by brown adipose tissue (BAT) can substantially contribute to weight loss and glucose metabolism [1][2]. The adipocytes of BAT have densely populated mitochondria containing uncoupling protein 1 (UCP1) which plays a pivotal role in thermogenesis [3]. Apart from classical BAT and WAT, there exists a third metabolically distinct cell type called beige adipocytes resulting from the browning of WAT in response to specific stimuli [4][5]. BAT and beige cells increase whole-body metabolism and energy expenditure via thermogenesis [6]. The activation of thermogenic adipocytes leads to body weight reduction and improved metabolism in animal models [7][8].

Dietary intake of some natural compounds can trigger thermogenic responses in experimental models, as well as humans [9]. Previously, plant-based small molecules such as berberine, butein, capsaicin, 7,8-Dihydroxyflavone and fucoxanthin have been reported to increase energy expenditure via stimulation of BAT [10-14]. Phytochemicals, including flavonoids and non-flavonoids, promote non-shivering thermogenesis thereby increasing energy expenditure [15][16].

Aframomum melegueta (Fam. Zingiberaceae) also known as Grains of Paradise (GP), is a native plant of West Africa. GP extracts have a pungent taste due to the presence of 6-paradol, an unsaturated non-volatile aromatic ketone [17]. Other pungent compounds in the extracts include 6-shogaol and 6-gingerol. GP seeds are used to relieve pain and inflammation in ethnomedicine. Several experimental studies have reported the potential anti-inflammatory and analgesic activities of GP in rats [18][19].

The inventor surprisingly discovered that the active constituents extracted from Aframomum melegueta, namely 6-paradol, 6-shogaol, and 6-gingerol, promote and activate BAT leading to thermogenic expenditure when the constituents are administered to a subject. The inventor discovered that this thermogenic expenditure can be harnessed to promote and support a healthy body weight, as well as support liver function and health, and a healthy lipid profile.

### SUMMARY OF THE INVENTION

It is an object of the invention to provide methods of promoting or maintaining a healthy body weight and supporting liver health and function, comprising administering to a person in need thereof an effective amount of a composition comprising 6-paradol.

In some aspects, the composition comprises between about 2% and about 15% 6-paradol.

In some aspects, the composition further comprises at least one of 6-shogaol and 6-gingerol.

In some aspects, the composition comprises about 8% 6-shogaol, about 17% 6-gingerol, or a combination thereof.

In some aspects, the composition is an extract of Aframomum melegueta seeds.

In some aspects, the composition is administered systemically, such as orally.

In some aspects, the composition is administered at a dose of about 250 mg.

In some aspects, the composition is administered two or more times per day.

In some aspects, the composition is combined with a food, snack, nutritional supplement, dietary supplement, food supplement, or beverage.

It is a further object of the invention to provide a composition for supporting liver health and function, comprising 6-paradol.

In some aspects, the composition further comprises 6-shogaol and 6-gingerol.

In some aspects, the composition comprises between about 2% and about 15% 6-paradol, about 8% 6-shogaol, and about 17% 6-gingerol.

In some aspects, the composition is an extract of Aframomum melegueta seeds.

In some aspects, the composition further comprises at least one of an artificial excipient, an artificial pharmaceutical carrier, an artificial bulking agent, an artificial binding agent, an artificial filler, and an artificial diluent.

In some aspects, the composition is combined with a food, snack, nutritional supplement, dietary supplement, nutraceutical, food supplement, or beverage.

It is a further object of the invention to provide a method of inducing the browning of white adipocytes into beige adipocytes in a subject in need thereof, comprising administering a composition as disclosed herein to the subject.

It is a further object of the invention to provide a method of treating obesity and other metabolic disorders in a subject in need thereof, comprising administering a composition as disclosed herein to the subject.

### BRIEF DESCRIPTION OF DRAWINGS

FIG. 1 is an HPLC chromatogram of an embodiment of the inventive composition showing peaks of 6-paradol, 6-Shogaol and 6-gingerol.

FIG. 2A is a graph showing the effect of an embodiment of the inventive composition on body weight (g) of high fat diet model mice.

FIG. 2B is a graph showing the body weight gain (g) of mice at the end of treatment from baseline.

FIG. 3A shows representative images of liver histology of high fat diet model mice.

FIG. 3B shows the effect of the administration an embodiment of the inventive composition on liver weight, and serum levels of AST and ALT in high fat diet model mice.

FIG. 4 demonstrates the effect of an embodiment of the inventive composition on serum lipid profile in high fat diet model mice.

FIG. 5A is a graph showing the effect of an embodiment of the inventive composition on mean weights of BAT in high fat diet model mice.

FIG. 5B is a Western blot analysis showing changes in the markers of BAT activity of high fat diet model mice.

FIG. 5C shows the quantitative analysis of expression of markers of BAT activity in high fat diet model mice.

FIG. 6A shows effect of an embodiment of the inventive composition on white adipose tissues (WAT) such as epidydimal and mesenteric fat depots in high fat diet model mice.

FIG. 6B shows H&E staining of epidydimal white adipose tissue (eWAT) histomorphology after 6 weeks of administration of an embodiment of the inventive composition in high fat diet model mice.

FIG. 6C is a graph showing the effect of an embodiment of the inventive composition on adipocyte size in eWAT of high fat diet model mice.

FIG. 6D shows the expression of BAT proteins in eWAT analyzed using Western blotting.

FIG. 6E shows the quantitative analysis of expression of BAT markers in WAT of high fat diet model mice.

FIG. 7 shows the participant flowchart in the clinical trial.

### DEFINITIONS

As used herein, the term “about” means the quantity, level, value, number, frequency, percentage, dimension, size, amount, weight or length that is referenced, or that varies (plus or minus) by as much as 30%, 25%, 20%, 15%, 10%, 9%, 8%, 7%, 6%, 5%, 4%, 3%, 2% or 1% to the referenced quantity, level, value, number, frequency, percentage, dimension, size, amount, weight or length.

As used herein, the phrases “effective amount,” “effective dose,” and “therapeutically effective amount” refer to that amount of an agent or composition that is sufficient to produce the targeted outcome, such, for example, promoting or supporting health or a bodily function or parameter, ameliorating a disorder, disease or condition, or the symptoms of a disorder, disease or condition.

As used herein, the terms “reduce,” “reducing,” and the like, refer to any measurable decrease in a parameter relative to control conditions.

As used herein, the terms “increase,” “increasing,” and the like, refer to any measurable increase in a parameter relative to control conditions.

As used herein, the term “treating” refers to reversing, preventing, alleviating or inhibiting the progress of a condition or disorder, or one or more symptoms of condition or disorder. As used herein, “treating” may also refer to decreasing the probability or incidence of the occurrence of a condition or disorder in a subject as compared to an untreated control population, or as compared to the same subject prior to treatment. For example, as used herein, “treating” may refer to preventing condition or disorder, and may include delaying or preventing the onset of condition or disorder, or delaying or preventing the symptoms associated with an condition or disorder. As used herein, “treating” may also refer to reducing the severity of a condition or disorder, or symptoms associated with such condition or disorder prior to affliction with the condition or disorder. Such prevention or reduction of the severity of a condition or disorder prior to affliction relates to the administration of the composition of the present technology, as described herein, to a subject that is not at the time of administration afflicted with the condition or disorder. As used herein “treating” may also further refer to preventing the recurrence of condition or disorder, or of one or more symptoms associated with such condition or disorder. The terms “treat,” “therapy,” “treatment,” and “therapeutically,” as used herein, refer to the act of treating as defined herein. Conditions and disorders treatable by the composition of the invention include obesity and metabolic disorders, such as diabetes (type 1 and type 2), hyperlipidemia, and hypercholesterolemia.

### DETAILED DESCRIPTION

The inventor surprisingly discovered 6-paradol, and other active ingredients, have efficacy in promoting thermogenesis through various physiological mechanisms, including the browning of WAT, promoting BAT, and promoting energy expenditure in these tissues.

Accordingly, the invention generally relates to a composition comprising 6-paradol as an effective ingredient for promoting thermogenesis and methods of using the composition in a variety of health applications. More particularly, the invention relates a composition comprising 6-paradol and methods of using and manufacturing the composition in health applications such as promoting and maintaining a healthy body weight and supporting the health and function of the liver.

In one non-limiting embodiment, the invention provides a composition comprising at least one of 6-paradol, 6-shogaol and 6-gingerol as active ingredients. In some aspects, the composition is standardized for one or more of these ingredients. The composition can be standardized to comprise, for example, up to about 5% 6-paradol, up to about 10% 6-paradol, up to about 12% 6-paradol, up to about 15% 6-paradol, up to about 20% 6-paradol, up to about 30% 6-paradol, up to about 40% 6-paradol, or up to about 50% 6-paradol. In some aspects, the composition is standardized to comprise between about 2% and about 12% 6-paradol, between about 2% and about 15% 6-paradol, between about 2% and about 20% 6-paradol, between about 2% and about 30% 6-paradol, between about 2% 6 and about 40% 6-paradol, or between about 2% and about 50% 6-paradol. In some aspects, the composition can consist essentially of 6-paradol, 6-shogaol and 6-gingerol according to the amounts disclosed herein.

In some embodiments, the composition is standardized for one or more of 6-shogaol and 6-gingerol. The composition can comprise up to about 2%, up to about 6%, up to about 8%, up to about 10%, up to about 15%, or up to about 20% 6-shogaol. In one non-limiting embodiment, the composition comprises between about 6% and about 8% 6-shogaol. The composition can be standardized for 6-gingerol, such that the composition comprises up to about 2%, up to about 5%, up to about 8%, up to about 10%, up to about 12%, up to about 15%, up to about 17%, up to about 20%, up to about 25%, or up to about 30% 6-gingerol. In one non-limiting embodiment, the composition comprises: between about 2% and about 15% 6-paradol; about 8% 6-shogaol; and about 17% 6-gingerol. In some embodiments, the composition comprises one or more of 6-paradol, 6-shogaol and 6-gingerol, wherein the 6-paradol, 6-shogaol and 6-gingerol are purified and are standardized for these components according to the amounts disclosed herein. As used herein, the term “purified” means the referenced agent or material is free of any other agent or material, or contains less than about 20%, less than about 10%, or less than about 5% of another agent or material.

In some non-limiting embodiments, the composition is an extract of Aframomum melegueta. The composition can be an extract of one or more of the seeds, leaves, stems, flowers, roots, and bark of Aframomum melegueta. The composition can be such an extract that is standardized for one or more of 6-paradol, 6-shogaol and 6-gingerol according to the amounts for these ingredients disclosed herein. For example, the extract can be an extract of Aframomum melegueta seeds that comprises: between about 2% and about 15% 6-paradol; about 8% 6-shogaol; and about 17% 6-gingerol. It will be understood that references to the percentage amounts of the ingredients of the composition herein refer to the weight-weight percentage (w/w %) amount of the referenced ingredient that is present in the composition. It will also be understood that the amounts of the components of the composition disclosed herein can be expressed in a ratio, wherein the ratio is determined according to the weight-weight percentages disclosed herein. In some aspects, the composition can consist essentially of an extract of Aframomum melegueta, wherein the extract is derived from one or more of the seeds, leaves, stems, flowers, roots, and bark of Aframomum melegueta according to the methods disclosed herein.

In some embodiments, the composition further comprises one or more substances selected from a vitamin, mineral, extract, amino acid, protein, carbohydrate, lipid, fatty acid, caffeine, flavouring, sweetener, preservative, and combinations thereof. In other aspects, the composition further comprises an excipient, pharmaceutically acceptable carrier, bulking agent, binding agent. film-forming agent, encapsulating agent, wall or shell material, matrix compound, coating, emulsifier, surface active agent, solubilizing agent (such as an oil, fat, wax, lecithin, and the like), adsorbent, filler, co-compound, dispersing agent, wetting agent, processing aid, flowing agent, taste-masking agent, weighting agent, jellyfying agent, gel-forming agent, antimicrobial, or combinations thereof. The composition can further comprise one or more artificial excipient, artificial pharmaceutically acceptable carrier, artificial bulking agent, or artificial binding agent. Suitable, excipients, pharmaceutically acceptable carriers, bulking agents, and binding agents include, but are not limited to, those disclosed in the following publications, the disclosures of which are incorporated herein by reference in their entirety for all purposes: Remington: The Science and Practice of Pharmacy, 19^{th} Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N.Y.: Marcel Decker 1980); and Pharmaceutical Dosage Forms and Drug Delivery Systems, Seventh Ed (Lippincott Williams & Wilkins 1999). The composition can be combined with, and administered as, a food, food supplement, beverage, or nutritional supplement.

In at least one embodiment, the composition can further comprise controlled, sustained, or extended-release formulations known collectively as “modified release” formulations. Modified-release formulations for use with the invention include, but are not limited to, protective hydrocolloid, such as gums, proteins, and modified starches, for example. Suitable modified release and delivery systems for use with the invention include, but are not limited to, those described in the following U.S. Patents, the entire disclosures of which are incorporated herein by reference for all purposes: 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566. Dosage forms for the composition can be used to provide modified release of one or more active ingredients using, for example, hydropropylmethyl cellulose, other polymer matrices, gels, permeable membranes, osmotic systems, multilayer coatings, microparticles, liposomes, microspheres, and combinations thereof.

In at least one aspect, the composition is provided and/or manufactured in bulk. The composition can be provided in bulk for the manufacture of foods, nutritional supplements, nutraceuticals, dietary supplements and/or food supplements. Bulk quantities of the composition can be packaged, stored and/or distributed in drums, bags, boxes, containers and the like. Such containers can be configured to prevent or inhibit the oxidation of the active ingredients of the composition.

At least one aspect of the invention concerns the form of the composition. The composition can be in made in a form selected from the group consisting of a powder, liquid, pill, tablet, pellet, capsule, thin film, solution, spray, syrup, linctus, lozenge, pastille, chewing gum, paste, vapor, suspension, emulsion, ointment, cream, lotion, liniment, gel, drop, topical patch, buccal patch, bead, gummy, gel, sol, injection, and combinations thereof. The composition can be formulated for systemic administration, such as, for example, oral administration.

In some aspects, the invention provides a method of making the composition. The composition can be made by combining one or more of 6-paradol, 6-shogaol and 6-gingerol. These ingredients can be purified. The ingredients can combined according to produce a composition that is standardized for the ingredients according to the amounts disclosed herein.

In some embodiments, the composition is an extract of Aframomum melegueta. Thus, the composition can be made by producing an extract from one or more of the seeds, leaves, stems, flowers, roots, or bark of Aframomum melegueta. These materials can be fresh, dried, partially dried, or a combination thereof. The materials can be whole, crushed, powdered, granulated, or a combination thereof. The extract can be produced by subjecting these materials to extraction using a suitable solvent. Suitable solvents for use with the invention include, but are not limited to, aqueous solvents, alcohol-based solvents, hydroalcoholic solvents, supercritical fluids, polar organic solvents (such as acetone and methylethyl ketone), and combinations thereof. Non-limiting examples of alcohol-based and hydroalcoholic solvents include, but are not limited to, ethanol, isopropyl alcohol, methanol, and combinations thereof. Suitable super-critical fluid solvents for use with the invention include, but are not limited to, carbon dioxide, oxygen, nitrogen, and combinations thereof.

In some embodiments, the invention provides methods of maintaining, supporting, or promoting the general health and well-being of a person in need thereof. In one non-limiting embodiment, the invention provides a method of promoting thermogenesis in a person in need thereof. Such methods can be practiced by administering the inventive composition to a person in need thereof. In one aspect, the invention provides a method of promoting or maintaining a healthy body weight, comprising administering to a person in need thereof an effective amount of the inventive composition. Administering the composition can reduce body weight, prevent an increase in body weight, or inhibit increases in body weight, in the person. In some aspects, the administration of the composition is combined with an exercise routine for promoting weight loss in the person. Without being limited to any particular theory or mechanism, administering the composition of the invention promotes the production of BAT tissue and BAT activity. This increase in BAT tissue and activity leads to increased thermogenic activity and the consumption of caloric energy leading to weight loss and the prevention of weight gain.

In other embodiments, the invention provides a method of supporting liver health and function in a person in need thereof, comprising administering to the person an effective amount of the inventive composition. In some aspects, the person consumes or has a history of consumption of at least one of drugs, alcohol, and a high fat diet. Administration of the composition can maintain or support healthy liver enzyme levels, including, without limitation, aspartate aminotransferase (AST) and alanine aminotransferase (ALT). Thus, a person in need of administration of the inventive composition can have elevated levels of at least one of AST and ALT. Moreover, the person in need of administration of the inventive composition can have compromised liver health or function as a result of hepatitis. In some aspects, the person is in need of administration of the inventive composition has jaundice, abdominal pain or swelling, swelling in the legs and ankles, itchy skin, dark urine, pale stool, chronic fatigue, nausea or vomiting, loss of appetite, tendency to bruise easily, or combinations thereof. Without being limited to any particular theory or mechanism, administering the composition can promote the health and function of the liver by supporting the production of bile, supporting the production of blood plasma proteins, supporting the production of cholesterol and apolipoproteins, supporting the conversion of excess glucose to glycogen, improving regulation of blood glucose, improving regulation of serum amino acid levels, improving the conversion of ammonia to urea, improving the clearance of drugs, alcohol and other toxins from the blood, improving regulation of blood clotting, improving resistance to infection, improving the clearance of bilirubin from the blood, or combinations thereof.

In some embodiments, the invention provides a method of treating a condition or disorder in a person in need thereof. The method can be practiced by administering to such person an effective amount of the inventive composition, wherein the administration to the person treats the targeted condition or disorder. In some aspects, the condition or disorder is obesity and administering the inventive composition results in weight loss or the prevention or inhibition of weight gain in the person. The person can be obese, or at risk of becoming obese. In other aspects, the condition is hyperlipidemia and administering the composition prevents an increase in, inhibits an increase in, or lowers, at least one of serum cholesterol and serum triglycerides in the person. The person can have hyperlipidemia, or be at risk of developing hyperlipidemia. In still other aspects, the condition is hepatic steatosis and administering the composition prevents an increase in, inhibits an increase in, or reduces lipids in the liver of the person. The person can have hepatic steatosis, or be at risk of developing hepatic steatosis.

The methods of the invention can be practiced by administering the composition at a selected dosage (i.e. effective amount). Suitable dosages of the composition include, but are not limited to, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, about 950 mg, or about 1,000 mg. The composition can be administered one, two, three, four, five, or more times per day. In a non-limiting embodiment, the composition is administered at a dose of about 500 mg per day. In a non-limiting preferred embodiment, the composition is administered at a dose of 250 mg twice a day.

At least one aspect of the invention concerns the administration route for the composition. Accordingly, the inventive composition can be administered systemically or non-systemically. Suitable administration routes for the composition include, but are not limited to, orally, buccally, sublingually, or a combination thereof In a non-limiting preferred embodiment, the composition is administered orally.

While the methods disclosed herein refer to administering the composition to a person (i.e. human), it will be appreciated that the methods can be practiced by administering the composition to a non-human subject. The subject can be a laboratory animal including, for example, a rodent such as mouse, rat, guinea pig, and the like.

References to the “composition” and the “inventive composition” are used interchangeably herein to refer to the composition of the present disclosure.

The foregoing detailed description and the accompanying drawings to which it refers are intended to describe some, but not necessarily all, examples, or embodiments of the invention. The described embodiments are to be considered in all respects only as illustrative and not restrictive.

### EXAMPLES

The inventive composition is exemplified below. Example 1 illustrates the quantitative analysis of an embodiment of the inventive composition by HPLC. Example 2 illustrates the anti-obesity effects of the composition of Example 1 in high fat diet-induced obesity model mice. Example 3 illustrates the thermogenic effects of the composition of Examples 1 and 2 in human subjects.

### Example 1

HPLC was performed on a Shimadzu LC2030 C Prominence-i (Japan) system equipped with a quaternary low-pressure gradient solvent delivery LC2030 pump with high-pressure switching valves, online LC2030 degasser unit, a high sensitivity LC2030 ultraviolet (UV) detector, high speed drive LC2030 autosampler and large capacity column oven. The system controlled and data analyzed by Lab Solutions software. A separation was carried out in Kinetex C-18 column (100 Å, 250 mm×4.6 mm, 5 μm pore size).

6-paradol in an embodiment of the inventive composition was quantitatively analysed at UV detection of 230 nm using a mobile phase of a mixture of orthophosphoric acid (0.2%): acetonitrile (40:60) with isocratic flow rate of 1.2 ml/min and the injection volume of 5 μl. For the analysis of 6-shogaol and 6-gingerol contents, a mobile phase of formic acid (0.5%): acetonitrile (10:90) with an isocratic flow rate of 0.5 ml/min was used. The compounds were quantified at UV detection of 280 nm. All solutions were degassed and filtered through 0.45 μm pore size filter. The column was maintained at 26 ° C. throughout the analysis. Methanol was used as a diluent and the total liquid chromatography (LC) run time was 20 min. The peaks of compounds in the composition were quantified by comparing with the retention time (RT) of respective reference standards separately.

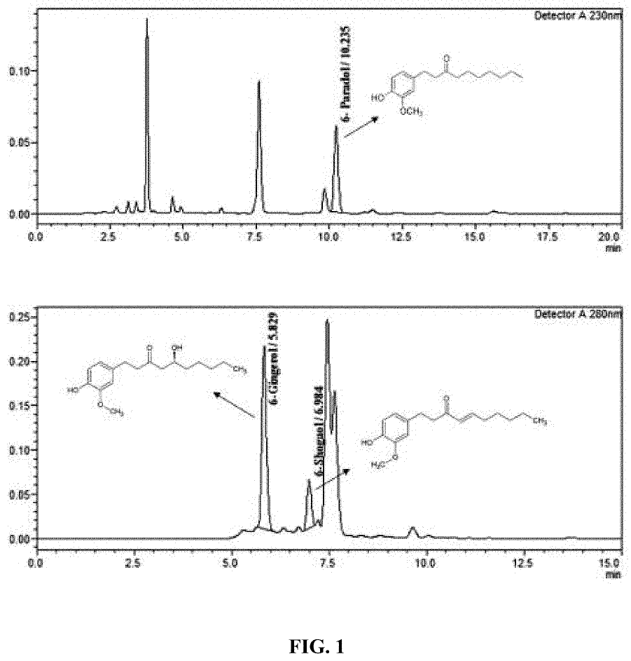

FIG. 1 shows the HPLC chromatograms of major active constituents in an embodiment of the inventive composition. HPLC analysis revealed the presence of 6-paradol, 6-shogaol and 6-gingerol with a content of 14.73%, 7.58% and 17.12% respectively. The chromatograms were confirmed using RT values of reference standards.

### Example 2

An embodiment of the inventive composition from Example 1 was demonstrated to support weight loss in a subject via activation of beige adipocytes and enhancing the brown adipose tissue activity.

Animals

Male C57B1/6 mice (12-week old) were procured from Hylasco Biotechnology Pvt Ltd. India (Reg No. 1808/PO/RcBt/S/15/CPCSEA). Mice were housed in a room under controlled temperature (22±3 ° C.), humidity (30-70%) with a 12 h light/dark cycle). All animals had access to food and water ad libitum. The animal experiments were conducted after approval from the Institutional Animal Ethics Committee (VHPL/PCL/IAEC/15/18 dated 21/09/2018) of Vidya Herbs Pvt Ltd.

After a one-week acclimatization, the animals were randomized into four groups (n=10 per group): control group (Control); High fat diet (HFD)-fed mice (HFD); HFD plus 20 mg/kg body weight composition (AF20); HFD plus 40 mg/kg body weight composition (AF40). The untreated and treated HFD mice were given HFD for 14 weeks. The mice in AF20 and AF40 were administered p.o with respective doses of the composition from week 9 to the end of week 14 (6 weeks in total). The animals were monitored for daily feed consumption. The body weight changes were recorded on weekly basis. After 14 weeks, all the mice were sacrificed using overdose of gaseous anesthesia (5% isoflurane). Blood samples were collected by heart puncture and centrifuged at 3500 rpm for 5 min to obtain serum. Liver and fat depots were harvested and weighed. WAT and BAT tissues were homogenized in tissue lysis buffer (50 mM Tris pH 7.5, 150 mM NaCl, 1% triton X-100) for further analysis.

Assessment of Body Weight Changes

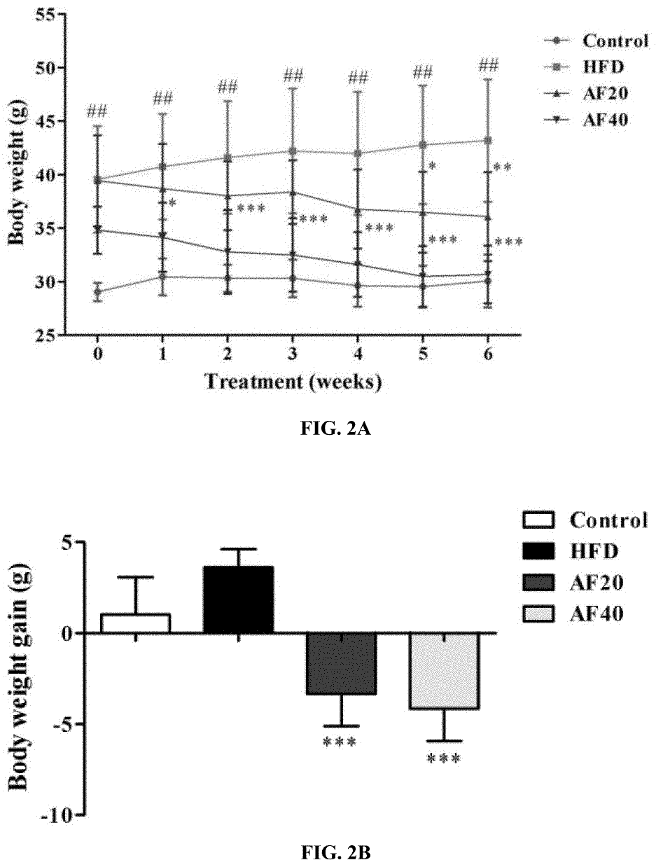

The initial body weight of animals in all the groups was similar at the start of experiment. 8-week consumption of HFD resulted in marked increase in the body weight of animals as compared to control group. The untreated HFD group mice gained significant weight up to 14 weeks (p<0.001). A 6-week administration of the composition dose-dependently reduced the body weight of HFD mice (FIG. 2A). The increase in body weight with respect to control group was 39.5%, 26.05% and 7.8% for HFD, AF20 and AF40 groups respectively. There was a significant reduction in the mean body weight of the high dose group (AF40) after one week of treatment and consistently decreased thereafter until the end of treatment (p<0.001) compared to the untreated HFD group. AF20 and AF40 treated groups showed a significant reduction in mean body weight change from the baseline as compared to HFD group (FIG. 2B).

Liver Histology

The histological changes in the liver were examined using hematoxylin and eosin (H&E) staining. Briefly, the tissues were fixed in 4% formalin and embedded in paraffin. The paraffin sections were then cut into 4 μm thickness and stained with H&E and observed for liver histomorphology. Similarly, the tissue samples were processed for Oil red O staining to examine the lipid droplets in liver. The images were captured using a microscope (Leica, Germany).

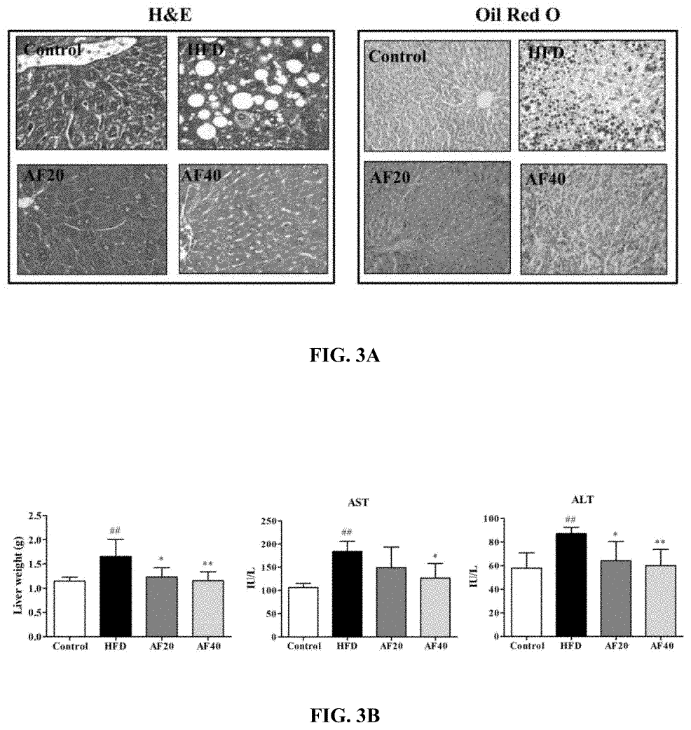

The untreated HFD mice showed the presence of macrovesicles and lipid droplets compared to the control group with a normal liver architecture. Low and high dose treatment with the composition restored the normal liver histology in HFD-mice. Oil red O staining showed considerable fat accumulation in the liver section of the HFD group. Treatment markedly reduced the hepatic lipid accumulation (FIG. 3A).

Liver Weight and Liver Function Parameters

The HFD mice exhibited a significant increase in the liver weight compared to the normal diet-fed animals (p<0.05). A 6-week administration of the composition at 20 and 40 mg/kg decreased the liver weight of HFD mice in a dose-dependent manner compared to the untreated HFD fed mice (FIG. 3B).

The HFD mice showed significant increase in the serum levels of AST (42.2%) and ALT (50.4%) as compared to control (p<0.01). Treatment with low and high doses of the composition resulted in a dose-dependent reduction in the levels of liver function parameters. The AF20 group showed a reduction of 18.76% and 26.44% in serum AST and ALT levels respectively compared with that of HFD group. The AF40 group exhibited a significant decrease of 31.05% and 31.03% respectively in AST (p<0.05) and ALT activities (p<0.01) as compared to untreated HFD mice (FIG. 3B).

Serum Biochemical Measurements

The serum samples were analysed for aspartate aminotransferase (AST), alanine aminotransferase (ALT), cholesterol, triglycerides (TG), low density lipoprotein-c (LDL-c) and high-density lipoprotein-c (HDL-c) using commercial kits from Randox Laboratory in a biochemical analyzer (Randox RX Imola, Co Antrim, UK).

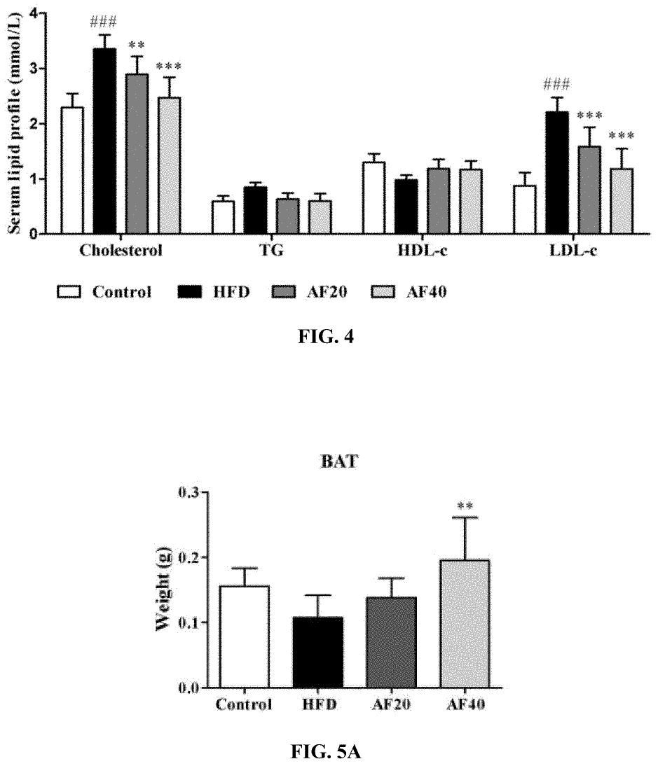

The untreated HFD mice showed significantly higher levels of total cholesterol and LDL-c (p<0.001) compared to control group. There was a marked increase in triglycerides (TG) and reduction in HDL-c observed in HFD group. However, low and high dose treatment groups (AF20 and AF40) had significantly reduced the TC and LDL-c (p<0.01) compared to untreated HFD group. The composition-treated groups dose-dependently exhibited improvement in the TG and HDL-c levels (FIG. 4).

Brown Adipose Tissue (BAT) Activity

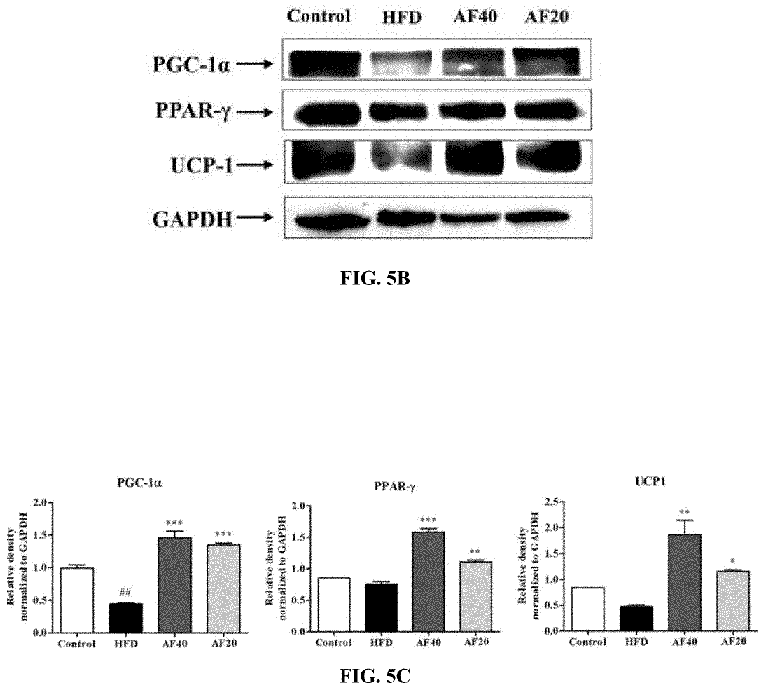

There was a dose-dependent increase in BAT weight following treatment with the composition. In the AF40 treatment group, the mean weight was significantly higher compared to untreated the HFD group (p<0.01) indicating improved BAT activity (FIG. 5A). Western blot analysis of BAT revealed that the composition treatment induced the expression of key proteins activating thermogenic program (FIGS. 5B and 5C). The expression of UCP-1, classical marker of BAT thermogenesis was significantly increased in AF20 and AF40 groups compared to the HFD group (p<0.01). The other transcription factors such as PGC-1α (p<0.001) and PPARγ (p<0.01) were strongly activated in low and high dose composition treatment groups. These results suggest that oral administration of the composition activate BAT activity in HFD mice.

Induction of Beige Adipocytes

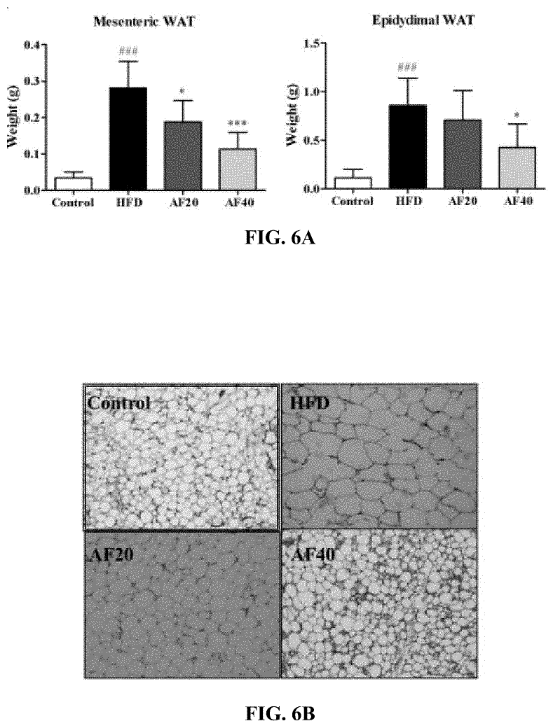

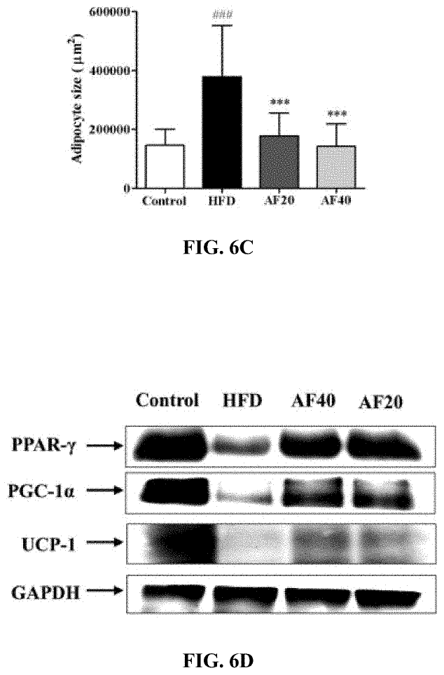

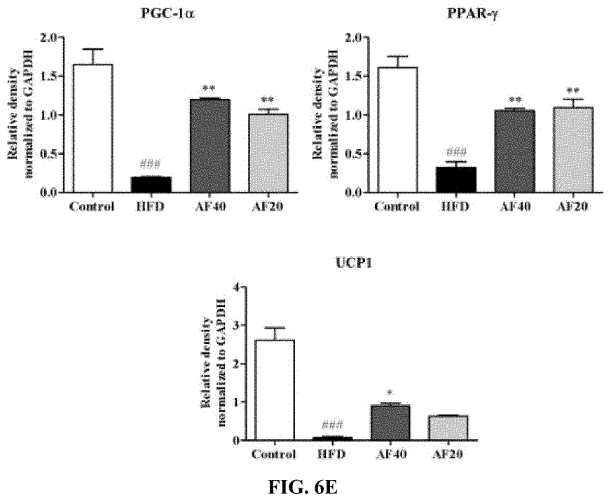

The mean weight of mesenteric and epidydimal WAT in the HFD group was significantly increased compared to the control group (p<0.001) (FIG. 6A). The AF20 and AF40 groups dose-dependently reduced the WAT weight of HFD mice. Consistent with reduced WAT mass, the HFD mice by composition treatment had significantly smaller adipocyte size compared to the untreated HFD group (p<0.001) (FIGS. 6B and C). These results correlate with the reduction in body weight of animals treated with respective doses of the composition.

Interestingly, the composition induced the markers of brown adipocytes in eWAT. It was observed that high doses of the composition (AF40) strongly induced the expression of PGC-1α and PPARγ (p<0.01) as compared to the untreated HFD group (FIGS. 6D and E). The expression of UCP-1 was increased in the composition-treated groups. However, the data were not significant compared to the untreated HFD group. These data suggest that the composition leads to browning of eWAT which can substantially contribute to its anti-obesity effects.

### Example 3

Ethics, Consent and Permissions

The Institutional Ethics Committee of Aman Hospital and Research Center, Vadodara, India approved the study protocol and informed consent form (AHRC/IEC/030/2018). This clinical study was registered in Clinical Trials Registry — India (CTRI/2019/03/017956 dated 07/03/2019).

Subjects

Seventy overweight (BMI≥25.0 to ≤30.0 kg/m²) male and female subjects aged 20-50 years were enrolled into this clinical trial. Before enrolment, subjects were explained the study protocol approved by the Institutional Ethics Committee, and informed consent and consent to publish were obtained.

Subjects suffering from chronic health conditions (e.g., diabetes, hypertension, chronic renal failure, heart and liver disease), fasting blood glucose above normal limits, history of chronic metabolic disease, psychiatric illness, drug abuse, smoking, addition to alcohol, endocrine abnormalities, history of surgery were excluded from the study.

Study Design

The clinical study to investigate the safety and efficacy of the composition was conducted in compliance with ICH-GCP (International Conference on Harmonization of Technical Requirements for Registration of Pharmaceuticals for Human Use—Good Clinical Practice) guidelines and Helsinki Declaration Standards. This clinical study adhered to the CONSORT guidelines.

A randomized, double-blind, placebo-controlled, two arm, single-centered clinical study was conducted in healthy overweight male and female human subjects. The study was performed at Aman Hospital and Research Center, Vadodara, Gujarat, India. A population size of 70 subjects were randomized to two treatment arms: the composition and placebo on 1:1 ratio (n=35 in each group). The subjects were randomized using block randomization. The investigator and the subjects were blinded of the interventions.

The subjects received oral doses of 250 mg capsules twice daily (before breakfast and dinner) for 12 weeks. The study treatment details are provided in Table 1. After the baseline/screening visit, two visits were scheduled at the study site during the intervention period at an interval of 6 weeks.

TABLE 1
Study treatment details

| | TREATMENT | TREATMENT |
|---|---|---|
| | GROUP A | GROUP B |
| Product | Composition | Maltodextrin |
| Dosage Form | Capsules | Capsules |
| Composition | Capsule weight - | Capsule weight - |
| | 250 mg | 250 mg |
| | Composition extract | Maltodextrin - |
| | powder - 250 mg | 250 mg |
| Route | Oral | Oral |
| Dose | 250 mg | 250 mg |
| Dosing | Twice a day, | Twice a day, |
| Regimen | before food | before food |
| Treatment | 84 days | 84 days |
| duration | | |
| Manufacturer | Vidya Herbs Pvt Ltd. | Vidya Herbs Pvt Ltd. |

Sample Size

A sample size of 70 subjects (35 per group) was considered enough to detect a clinically important difference between groups with 80% power and a 5% level of significance. Considering an estimated potential dropout rate of 12%, the sample size was finalized as 70 (35 per group).

Study Outcomes

The primary endpoints included efficacy parameters such as energy expenditure (indirect calorimetry) and body fat composition (Dual energy X-ray Absorptiometry (DEXA) and Computed tomography (CT scan)) measured at baseline and the end of study.

The secondary endpoint analysis included safety assessments (serum lipid profile, blood biochemical parameters and complete blood count) and the effect of the composition on the quality of life. To assess the quality of life, the 12-term Short Form Survey (SF-12) questionnaire was used.

Statistical Analysis

The data were analyzed by paired and independent sample t-test and the values presented as mean±SD. Data were considered statistically significant at p<0.05.

Results

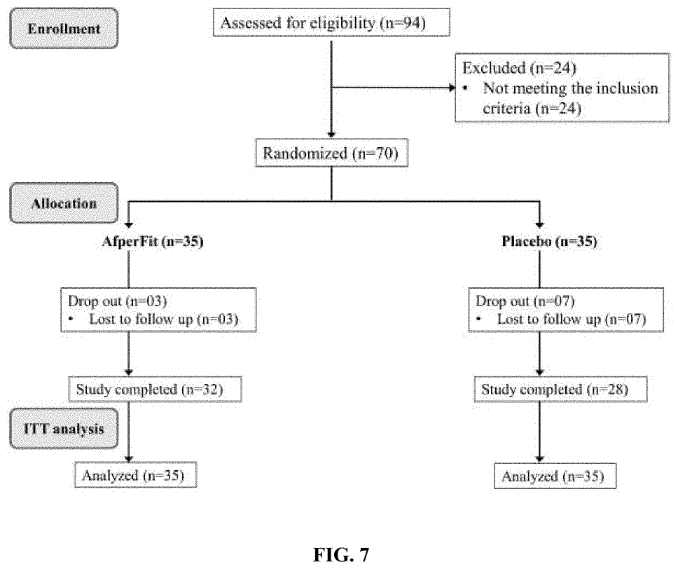

A total of 94 human volunteers were screened of which 70 subjects meeting the inclusion criteria were enrolled in the study. Of the 70 subjects randomized to two treatment groups (n=35 in each group), 60 subjects completed the study. There were 10 dropouts in the study (reason: lost to follow up). The intention-to-treat (ITT) analysis was used to assess the outcome of the study. The participant flow through the study is presented in FIG. 7. The demographic characteristics of subjects between the groups were not significantly different (Table 2). The efficacy and safety analysis were performed using ITT population.

TABLE 2
Demographic characteristics of subjects

| | Group A | Group B | |
|---|---|---|---|
| | (Composition) | (Placebo) | |
| Variable | (N = 35) | (N = 35) | p-value‡ |
| Age (Years) | 36.57 ± 9.22 | 36.05 ± 7.97 | 0.800‡ |
| Height (cm) | 161.47 ± 9.81 | 158.43 ± 8.55 | 0.171‡ |
| Weight (kg) | 72.78 ± 9.54 | 69.17 ± 7.27 | 0.079‡ |
| BMI (kg/m²) | 27.84 ± 1.73 | 27.54 ± 1.73 | 0.467‡ |

N: No of subjects;
BMI: Body Mass Index;
Date presented as: Mean ± Standard deviation
‡p values were Independent sample t-test;
p < 0.05 was considered as significant.

Efficacy Analysis

A 12-week treatment with the composition significantly increased the energy expenditure from baseline (1128.94±232.69 kcal/day) to the end of study (1338.31±297.89 kcal/day). The mean change in energy expenditure of the composition group was significant as compared to placebo group (p<0.01) (Table 3).

TABLE 3
Summary of energy expenditure (kcal/day) (Indirect calorimetry)

| | Composition, Group A | | Placebo, Group B | | |
|---|---|---|---|---|---|
| | (N = 35) | | (N = 35) | | ‡p value |
| Visit | N | Mean ± SD | N | Mean ± SD | between groups |
| V 1 | 35 | 1128.94 ± 232.69 | 35 | 1212.43 ± 349.24 | 0.2439‡ |
| V 3 | 35 | 1338.31 ± 287.89 | 35 | 1252.69 ± 331.14 | 0.2524‡ |
| Change | 35 | −209.37 ± 209.74 | 35 | −40.26 ± 231.90 | 0.0021‡* |
| V 1 vs. V 3 | | | | | |

| | | |
|---|---|---|
| #p value | <0.001#** | 0.312# |
| V 1 vs. V 3 | | |

N = No of subjects;
SD: Standard Deviation
‡p: Independent sample t-test;
#p: Paired sample t-test
V1: Visit 1, baseline;
V3: Visit 3, end of the study
*p < 0.01 and
**p < 0.001

The body composition was measured using DEXA analysis (Table 4). A significant reduction in body fat (%) was noticed in both the groups (p<0.01). The body fat reduction from baseline to the end of treatment was slightly superior in the composition group. There was a significant decrease in tissue mass and fat mass observed in the treatment groups. The mean changes in from baseline to the end of study were higher in the composition group as compared to placebo. Interestingly, a decreasing trend was observed in lean mass among the subjects in the composition group from baseline to the end of treatment (p<0.01). The placebo group showed an increase in lean mass (p<0.05).

Another aspect of the primary end point analysis was to examine the effect of the composition on body fat distribution. CT scan was used to measure the visceral and subcutaneous fat area. Table 5 shows the summary of fat distribution in the subjects. After 12-week supplementation of the composition, there was a significant decrease (p<0.01) in the visceral fat area of the subjects from baseline (71.42±31.12 cm²) to the end of treatment (54.83±28.42). On the contrary, a significant increase from baseline (66.42±33.85) to the end of treatment (86.25±59.00) in visceral fat area was noticed in the placebo group (p<0.05). The mean change from baseline to the end of treatment in the composition group (16.59±28.14) was significant (p<0.001) as compared to placebo (-19.83±50.54). The subcutaneous fat area was reduced in both the groups significantly (p<0.05). The mean change in subcutaneous fat area between the composition and placebo groups were not significant. These results clearly indicate the potential of the composition to specifically reduce the visceral fat.

TABLE 4
Summary of body fat composition (DEXA analysis)

| | Composition, Group A | | Placebo, Group B | | ‡p value |
|---|---|---|---|---|---|
| | (N = 35) | | (N = 35) | | between |
| Parameter | N | Mean ± SD | N | Mean ± SD | groups |

Body fat (%)

| | | | | | |
|---|---|---|---|---|---|
| V 1 | 35 | 43.52 ± 6.83 | 35 | 44.46 ± 7.54 | 0.5874‡ |
| V 3 | 35 | 40.68 ± 8.44 | 35 | 42.32 ± 7.92 | 0.4032‡ |
| Change | 35 | 2.84 ± 4.75 | 35 | 2.13 ± 4.20 | 0.5106‡ |
| V 1 vs. V 3 | | | | | |

| | | |
|---|---|---|
| #p value | 0.0012#** | 0.0049#** |
| V 1 vs. V 3 | | |

Tissue mass (kg)

| | | | | | |
|---|---|---|---|---|---|
| V 1 | 35 | 69.51 ± 9.36 | 35 | 65.72 ± 7.46 | 0.0657‡ |
| V 3 | 35 | 64.86 ± 10.97 | 35 | 64.74 ± 6.82 | 0.9552‡ |
| Change | 35 | 4.65 ± 5.45 | 35 | 0.98 ± 2.46 | <0.001‡*** |
| V 1 vs. V 3 | | | | | |

| | | |
|---|---|---|
| #p value | <0.001#*** | 0.024#* |
| V 1 vs. V 3 | | |

Fat mass (kg)

| | | | | | |
|---|---|---|---|---|---|
| V 1 | 35 | 30.18 ± 6.00 | 35 | 29.06 ± 5.35 | 0.4149‡ |
| V 3 | 35 | 26.67 ± 8.27 | 35 | 27.27 ± 5.40 | 0.7213‡ |
| Change | 35 | 3.51 ± 4.66 | 35 | 1.79 ± 3.51 | 0.0870‡ |
| V 1 vs. V 3 | | | | | |

| | | |
|---|---|---|
| #p value | <0.001#*** | 0.005#** |
| V 1 vs. V 3 | | |

Lean mass (kg)

| | | | | | |
|---|---|---|---|---|---|
| V 1 | 35 | 39.33 ± 7.5 | 35 | 36.51 ± 6.89 | 0.1064‡ |
| V 3 | 35 | 38.19 ± 7.3 | 35 | 37.32 ± 6.82 | 0.6092‡ |
| Change | 35 | 1.14 ± 2.47 | 35 | −0.81 ± 2.25 | 0.0010‡ |
| V 1 vs. V 3 | | | | | |

| | | |
|---|---|---|
| #p value | 0.0098#** | 0.041#* |
| V 1 vs. V 3 | | |

Fat free mass (kg)

| | | | | | |
|---|---|---|---|---|---|
| V 1 | 35 | 38.71 ± 13.64 | 35 | 30.87 ± 15.85 | 0.0299‡ |
| V 3 | 35 | 37.56 ± 13.25 | 35 | 31.71 ± 16.26 | 0.1035‡ |
| Change | 35 | 1.15 ± 2.606 | 35 | −0.84 ± 2.30 | 0.0012‡ |
| V 1 vs. V 3 | | | | | |

| | | |
|---|---|---|
| #p value | 0.013#* | 0.04#* |
| V 1 vs. V 3 | | |

N = No of subjects;
SD: Standard Deviation
‡p: Independent sample t-test;
#p: Paired sample t-test
V1: Visit 1, baseline;
V3: Visit 3, end of study
*p < 0.05,
**p < 0.01 and
***p < 0.001

TABLE 5
Summary of fat distribution assessment

| | Composition, Group A | | Placebo, Group B | | ‡p value |
|---|---|---|---|---|---|
| | (N = 35) | | (N = 35) | | between |
| Parameter | N | Mean ± SD | N | Mean ± SD | groups |

Visceral fat area (cm²)

| | | | | | |
|---|---|---|---|---|---|
| V 1 | 35 | 71.42 ± 31.12 | 35 | 66.42 ± 33.85 | 0.522‡ |
| V 3 | 35 | 54.83 ± 28.42 | 35 | 86.25 ± 59.00 | 0.006‡** |
| Change | 35 | 16.59 ± 28.14 | 35 | −19.83 ± 50.54 | <0.001‡*** |
| V 1 vs. V 3 | | | | | |

| | | |
|---|---|---|
| #p value | 0.0014#** | 0.026#* |
| V 1 vs. V 3 | | |

Subcutaneous fat area (cm²)

| | | | | | |
|---|---|---|---|---|---|
| V 1 | 35 | 703.51 ± 83.12 | 35 | 753.97 ± 81.70 | 0.013‡* |
| V 3 | 35 | 663.70 ± 115.38 | 35 | 705.99 ± 103.22 | 0.111‡ |
| Change | 35 | 39.81 ± 110.87 | 35 | 47.98 ± 110.38 | 0.758‡ |
| V 1 vs. V 3 | | | | | |

| | | |
|---|---|---|
| #p value | 0.041#* | 0.015#* |
| V 1 vs. V 3 | | |

N = No of subjects;
SD: Standard Deviation
‡p: Independent sample t-test;
#p: Paired sample t-test
V1: Visit 1, baseline;
V3: Visit 3, end of study
*p < 0.05,
**p < 0.01 and
***p < 0.001

The secondary end point analysis included recording of body weight and BMI (Table 6). As analysed from DEXA, the composition treatment group subjects showed significant reduction in the body weight and BMI from baseline to the end of treatment (p<0.001). The mean changes in body weight and BMI from baseline was significant in the composition group as compared to placebo (p<0.01).

TABLE 6
Summary of changes in body weight and body mass index (BMI)

| | Composition, Group A | | Placebo, Group B | | |
|---|---|---|---|---|---|
| | (N = 35) | | (N = 35) | | ‡p value |
| Parameter | N | Mean ± SD | N | Mean ± SD | between groups |

Body weight (kg)

| | | | | | |
|---|---|---|---|---|---|
| V 1 | 35 | 72.78 ± 9.54 | 35 | 69.17 ± 7.27 | 0.0797‡ |
| V 3 | 35 | 68.71 ± 11.28 | 35 | 68.36 ± 6.85 | 0.8754‡ |
| Change | 35 | 4.07 ± 5.45 | 35 | 0.81 ± 2.03 | 0.0018‡** |
| V 1 vs. V 3 | | | | | |

| | | |
|---|---|---|
| #p value | <0.001#*** | 0.024#* |
| V 1 vs. V 3 | | |

BMI (kg/m²)

| | | | | | |
|---|---|---|---|---|---|
| V 1 | 35 | 27.84 ± 1.73 | 35 | 27.54 ± 1.73 | 0.467‡ |
| V 3 | 35 | 26.19 ± 2.71 | 35 | 27.22 ± 1.93 | 0.072‡ |
| Change | 35 | 1.65 ± 2.16 | 35 | 0.32 ± 0.81 | 0.0013‡** |
| V 1 vs. V 3 | | | | | |

| | | |
|---|---|---|
| #p value | <0.001#*** | 0.026#* |
| V 1 vs. V 3 | | |

N = No of subjects;
SD: Standard Deviation
‡p: Independent sample t-test;
#p: Paired sample t-test
V1: Visit 1, baseline;
V3: Visit 3, end of study
*p < 0.05,
**p < 0.01 and
***p < 0.001

The effect of composition administration on general health was determined using quality of life questionnaire (SF-12). The objective assessment included total SF-12 score, physical component score (PCS-12) and mental component score (MCS-12). SF-12 total score in the composition group was significantly increased from baseline (29.40±1.58) to the end of study (31.69±2.10) (p<0.001). The change in the total score of the composition group (−2.29±1.76) was found to be significant (p<0.001) as compared to placebo (0.20±2.70). A similar trend was observed in MCS-12 score. However, there was no significant change in the PCS-12 score of the treatment groups (Table 7).

To summarize the efficacy measures, a 12-week oral supplementation of 500 mg/day of the composition could exert thermogenic effect by significantly increasing the energy expenditure and reducing the body fat % and visceral fat. There was an obvious reduction in the body weight and BMI of the subjects in the composition group.

TABLE 7
Summary of SF-12 scoring by visit and treatment

| | Composition, Group A | | Placebo, Group B | | ‡p value |
|---|---|---|---|---|---|
| | (N = 35) | | (N = 35) | | between |
| Parameter | N | Mean ± SD | N | Mean ± SD | groups |

Total score

| | | | | | |
|---|---|---|---|---|---|
| V 1 | 35 | 29.40 ± 1.58 | 35 | 30.03 ± 2.12 | 0.164‡ |
| V 3 | 35 | 31.69 ± 2.10 | 35 | 29.83 ± 2.67 | 0.002‡ |
| Change | 35 | −2.29 ± 1.76 | | 0.20 ± 2.70 | <0.001‡** |
| V 1 vs. V 3 | | | | | |
| #p value | | <0.001#** | | 0.664 | |
| V 1 vs. V 3 | | | | | |

PCS-12 score

| | | | | | |
|---|---|---|---|---|---|
| V 1 | 35 | 12.60 ± 1.50 | 35 | 12.77 ± 1.31 | 0.612‡ |
| V 3 | 35 | 12.57 ± 1.42 | 35 | 12.89 ± 1.43 | 0.359‡ |
| Change | 35 | 0.03 ± 1.10 | | −0.11 ± 1.53 | 0.655‡ |
| V 1 vs. V 3 | | | | | |
| #p value | | 0.879# | | 0.661# | |
| V 1 vs. V 3 | | | | | |

MCS-12 score

| | | | | | |
|---|---|---|---|---|---|
| V 1 | 35 | 16.80 ± 1.73 | 35 | 17.26 ± 1.62 | 0.257 |
| V 3 | 35 | 19.11 ± 1.41 | 35 | 16.94 ± 2.03 | <0.001‡** |
| Change | 35 | −2.31 ± 1.98 | 35 | 0.31 ± 2.26 | <0.001‡** |
| V 1 vs. V 3 | | | | | |
| #p value | | <0.001#** | | 0.416 | |
| V 1 vs. V 3 | | | | | |

N = No of subjects;
SD: Standard Deviation
‡p: Independent sample t-test;
#p: Paired sample t-test
V1: Visit 1, baseline;
V3: Visit 3, end of study
**p < 0.001

Safety Evaluation

Liver function and renal function parameters were evaluated at baseline and the end of the study. The parameters were found to be within the normal range throughout the study. No clinically significant difference in the biochemical parameters were observed between the groups at the end of study (Table 8).

Serum lipid parameters were found to be within normal levels throughout the study period. A significant (p<0.05) reduction from baseline in total cholesterol, LDL cholesterol, and LDL: HDL ratio was observed in the composition group. No clinically significant difference was found between the composition and placebo groups (Table 9).

Table 10 shows the hematological assessment of subjects. In the composition group, there were marginal variations in the parameters except for significant increase in the mean cell volume (MCV) and mean cell hemoglobin (MCH) (p<0.05).

There were no serious adverse events recorded during the study. The vital signs such as body temperature, pulse rate, systolic and diastolic blood pressure were within normal levels throughout the study. Overall, the composition was well tolerated when supplemented for 12 weeks at 500 mg/day.

TABLE 8
Serum biochemical analysis

| | Composition, Group A | | Placebo, Group B | | ‡p value |
|---|---|---|---|---|---|
| | (N = 35) | | (N = 35) | | between |
| Parameter | N | Mean ± SD | N | Mean ± SD | groups |

Aspartate aminotransferase (U/L)

| | | | | | |
|---|---|---|---|---|---|
| V 1 | 35 | 25.63 ± 4.95 | 35 | 26.60 ± 4.89 | 0.411‡ |
| V 3 | 35 | 24.14 ± 4.52 | 35 | 24.70 ± 4.82 | 0.617‡ |
| Change | 35 | 1.49 ± 4.33 | | 1.90 ± 4.67 | 0.703‡ |
| V 1 vs. V 3 | | | | | |
| #p value | | 0.05 | | 0.02#* | |
| V 1 vs. V 3 | | | | | |

Alanine aminotransferase (U/L)

| | | | | | |
|---|---|---|---|---|---|
| V 1 | 35 | 24.11 ± 5.94 | 35 | 25.50 ± 5.74 | 0.321‡ |
| V 3 | 35 | 26.21 ± 5.35 | 35 | 26.56 ± 5.78 | 0.795‡ |
| Change | 35 | −2.10 ± 4.31 | 35 | −1.05 ± 4.75 | 0.338‡ |
| V 1 vs. V 3 | | | | | |
| #p value | | 0.007#** | | 0.198# | |
| V 1 vs. V 3 | | | | | |

Alkaline phosphatase (U/L)

| | | | | | |
|---|---|---|---|---|---|
| V 1 | 35 | 89.01 ± 17.60 | 35 | 91.22 ± 16.05 | 0.586‡ |
| V3 | 35 | 88.66 ± 16.46 | 35 | 90.23 ± 15.56 | 0.683‡ |
| Change | 35 | 0.35 ± 11.59 | 35 | 0.99 ± 7.10 | 0.783‡ |
| V 1 vs. V 3 | | | | | |
| #p value | | 0.858# | | 0.415# | |
| V 1 vs. V 3 | | | | | |

Serum creatinine (mg/dL)

| | | | | | |
|---|---|---|---|---|---|
| V 1 | 35 | 0.95 ± 0.11 | 35 | 0.98 ± 0.13 | 0.206 |
| V 3 | 35 | 0.96 ± 0.10 | 35 | 1.01 ± 0.13 | 0.076 |
| Change | 35 | −0.01 ± 0.09 | 35 | −0.03 ± 0.09 | 0.550 |
| V 1 vs. V 3 | | | | | |
| #p value | | | | | |
| V 1 vs. V 3 | | | | | |

N = No of subjects;
U/L: Units/Litre;
mg/dl: Milligrams/Decilitre
#Paired t-test (V1 vs V3);
‡Independent t-test
*p < 0.05,
**p < 0.01

TABLE 9
Serum lipid profile

| | Composition, Group A | | Placebo, Group B | | ‡p value |
|---|---|---|---|---|---|
| | (N = 35) | | (N = 35) | | between |
| Parameter | N | Mean ± SD | N | Mean ± SD | groups |

Total cholesterol (mg/dL)

| | | | | | |
|---|---|---|---|---|---|
| V 1 | 35 | 197.84 ± 40.63 | 35 | 188.43 ± 41.07 | 0.338‡ |
| V 3 | 35 | 191.01 ± 39.81 | 35 | 185.59 ± 38.18 | 0.563‡ |
| Change | 35 | 6.83 ± 18.68 | 35 | 2.83 ± 11.53 | 0.286‡ |
| V 1 vs. V 3 | | | | | |
| #p value | | 0.038#* | | 0.155 | |
| V 1 vs. V 3 | | | | | |

Triglycerides (mg/dL)

| | | | | | |
|---|---|---|---|---|---|
| V 1 | 35 | 138.59 ± 28.03 | 35 | 136.17 ± 33.18 | 0.742‡ |
| V 3 | 35 | 138.93 ± 27.85 | 35 | 133.50 ± 30.38 | 0.439‡ |
| Change | 35 | −0.34 ± 14.84 | 35 | 2.66 ± 11.68 | 0.350‡ |
| V 1 vs. V 3 | | | | | |
| #p value | | 0.893# | | 0.186# | |
| V 1 vs. V 3 | | | | | |

Low density lipoprotein cholesterol (LDL-c) (mg/dL)

| | | | | | |
|---|---|---|---|---|---|
| V 1 | 35 | 128.14 ± 38.06 | 35 | 122.02 ± 43.64 | 0.534‡ |
| V 3 | 35 | 120.91 ± 37.75 | 35 | 116.65 ± 34.39 | 0.623‡ |
| Change | 35 | 7.22 ± 18.85 | 35 | 5.37 ± 23.18 | 0.715‡ |
| V 1 vs. V 3 | | | | | |
| #p value | | 0.03#* | | 0.180 | |
| V 1 vs. V 3 | | | | | |

High density lipoprotein cholesterol (HDL-c) (mg/dL)

| | | | | | |
|---|---|---|---|---|---|
| V 1 | 35 | 41.95 ± 1.66 | 35 | 42.01 ± 1.94 | 0.885‡ |
| V 3 | 35 | 42.61 ± 2.23 | 35 | 42.32 ± 1.36 | 0.521‡ |
| Change | 35 | −0.66 ± 2.05 | 35 | −0.66 ± 2.05 | 0.427‡ |
| V 1 vs. V 3 | | | | | |
| #p value | | 0.067# | | 0.250# | |
| V 1 vs. V 3 | | | | | |

Very low density lipoprotein (mg/dL)

| | | | | | |
|---|---|---|---|---|---|
| V 1 | 35 | 27.72 ± 5.61 | 35 | 27.23 ± 6.63 | 0.739‡ |
| V 3 | 35 | 27.79 ± 5.57 | 35 | 26.76 ± 6.13 | 0.466‡ |
| Change | 35 | −0.07 ± 2.97 | 35 | 0.47 ± 2.26 | 0.398‡ |
| V 1 vs. V 3 | | | | | |
| #p value | | 0.892# | | 0.228# | |
| V 1 vs. V 3 | | | | | |

LDL:HDL ratio

| | | | | | |
|---|---|---|---|---|---|
| V 1 | 35 | 3.07 ± 0.95 | 35 | 2.90 ± 0.98 | 0.485‡ |
| V 3 | 35 | 2.87 ± 0.94 | 35 | 2.79 ± 0.88 | 0.710‡ |
| Change | | 0.20 ± 0.47 | | 0.12 ± 0.44 | 0.463‡ |
| V 1 vs. V 3 | | | | | |
| #p value | | 0.018#* | | 0.119 | |
| V 1 vs. V 3 | | | | | |

N = No of subjects;
U/L: Units/Litre;
mg/dl: Milligrams/Decilitre
#Paired t-test (V1 vs V3);
‡Independent t-test
*p < 0.05,
**p < 0.01a

TABLE 10
Hematological assessment during the study

| | | Group A (Composition) | | | Group B (Placebo) | | | |
|---|---|---|---|---|---|---|---|---|
| | | (N = 35) | | | (N = 35) | | | |
| | | | | p value # | | | p value # | |
| | | | | (Baseline | | | (Baseline | p‡ |
| Parameters | Visits | Mean | SD | vs. EOS) | Mean | SD | vs. EOS) | (A Vs B) |
| Total | Baseline | 7374.29 | 1520.52 | 0.509 | 7822.86 | 1473.91 | 0.139 | 0.214 |
| Leukocyte | EOS | 7491.43 | 1415.75 | | 7554.29 | 1430.83 | | 0.854 |
| Count (cmm) | Change | −117.14 | 1038.52 | | 268.57 | 1049.59 | | 0.127 |
| Red Blood | Baseline | 5.09 | 0.49 | 0.128 | 4.99 | 0.32 | 0.094 | 0.321 |
| Cell count | EOS | 4.92 | 0.59 | | 4.93 | 0.27 | | 0.886 |
| (million/cmm) | Change | 0.18 | 0.66 | | 0.06 | 0.21 | | 0.341 |
| Hemoglobin | Baseline | 13.51 | 0.68 | 0.049 | 13.33 | 0.56 | 0.447 | 0.231 |
| (g/dL) | EOS | 13.38 | 0.51 | | 13.28 | 0.42 | | 0.390 |
| | Change | 0.13 | 0.39 | | 0.05 | 0.40 | | 0.380 |
| Hematocrit | Baseline | 40.65 | 1.60 | 0.217 | 39.96 | 1.54 | 0.888 | 0.071 |
| (%) | EOS | 40.29 | 1.38 | | 40.00 | 1.29 | | 0.370 |
| | Change | 0.36 | 1.68 | | −0.04 | 1.79 | | 0.338 |
| Mean Cell | Baseline | 80.37 | 6.68 | 0.010* | 80.21 | 4.33 | 0.231 | 0.904 |
| Volume (fL) | EOS | 83.55 | 4.47 | | 81.03 | 4.04 | | 0.016 |
| | Change | −3.17 | 6.89 | | −0.83 | 4.00 | | 0.087 |
| Mean Cell | Baseline | 26.71 | 2.24 | 0.0116* | 26.76 | 1.36 | 0.330 | 0.903 |
| Hemoglobin | EOS | 27.73 | 1.72 | | 26.97 | 1.18 | | 0.035 |
| (MCH) (Pg) | Change | −1.02 | 2.27 | | −0.21 | 1.23 | | 0.067 |
| MCH | Baseline | 33.25 | 1.20 | 0.9192 | 33.38 | 0.97 | 0.4975 | 0.609 |
| Concentration | EOS | 33.22 | 1.05 | | 33.23 | 1.16 | | 0.983 |
| (%) | Change | 0.03 | 1.49 | | 0.15 | 1.33 | | 0.705 |
| Platelet | Baseline | 300228.60 | 54702.45 | 0.0418 | 283857.10 | 43095.75 | 0.5554 | 0.169 |
| Count | EOS | 282714.30 | 46625.27 | | 279685.70 | 52932.13 | | 0.801 |
| (cmm) | Change | 17514.29 | 48992.42 | | 4171.43 | 41437.75 | | 0.223 |

N = No of subjects;
EOS: End of study;
cm³: cubic millimeter;
g/dl: Gram/deciliter
fL: femtolitres, 10^{-15 L};
Pg: picograms;
#: Paired t-test (baseline vs End of study);
‡Independent t-test

### REFERENCES

1. Kajimura S, Saito M. A new era in brown adipose tissue biology: molecular control of brown fat development and energy homeostasis. Annu Rev Physiol 2014; 76:225-249.
2. Poher A L, Altirriba J, Veyrat-Durebex C, Rohner-Jeanrenaud F. Brown adipose tissue activity as a target for the treatment of obesity/insulin resistance. Front Physiol 2015; 6: 4.
3. Rousset S, Alves-Guerra M C, Mozo J, Miroux B, Cassard-Doulcier A M, Bouillaud F. et al., The biology of mitochondrial uncoupling proteins. Diabetes 2014; 53(Suppl. 1): S130-S135.
4. Barbagallo I, Vanella L, Cambria M T, Tibullo D, Godos J, Guarnaccia L. et al., Silibinin regulates lipid metabolism and differentiation in functional human adipocytes. Front Physiol 2015; 6: 309.
5. Cohen P, Spiegelman B M. Brown and beige fat: molecular parts of a thermogenic machine. Diabetes 2015; 64:2346-2351.
6. Wu J, Cohen P, Spiegelman B M. Adaptive thermogenesis in adipocytes: is beige the new brown? Genes Dev 2013; 27(3):234-50.
7. Harms M, Seale P. Brown and beige fat: development, function and therapeutic potential. Nat Med 2013; 19(10):1252-63.
8. Seale P, Conroe H M, Estall J, Kajimura S, Frontini A, Ishibashi J. et al., Prdm16 determines the thermogenic program of subcutaneous white adipose tissue in mice. J Clin Investig 2011; 121(1):96-105.
9. Okla M, Kim J, Koehler K, Chung S. Dietary factors promoting brown and beige fat development and thermogenesis. Adv Nutr 2017; 8(3):473-483.
10. Zhang Z, Zhang H, Li B, Meng X, Wang J, Zhang Y, et al., Berberine activates thermogenesis in white and brown adipose tissue. Nat Commun 2014; 5:5493.
11. Song N J, Choi S, Rajbhandari P, Chang S H, Kim S, Vergnes L, et al., Prdm4 induction by the small molecule butein promotes white adipose tissue browning. Nat Chem Biol 2016; 12(7):479-81.
12. Luo Z, Ma L, Zhao Z, He H, Yang D, Feng X, et al., TRPV1 activation improves exercise endurance and energy metabolism through PGC-1 alpha upregulation in mice. Cell Res 2012; 22:551-564.
13. Liu C Y, Chan C B, Ye K Q. 7,8-dihydroxyflavone, a small molecular TrkB agonist, is useful for treating various BDNF-implicated human disorders. Transl Neurodegener 2016; 5:2.
14. Maeda H, Hosokawa M, Sashima T, Funayama K, Miyashita K. Fucoxanthin from edible seaweed, Undaria pinnatifida, shows antiobesity effect through UCP1 expression in white adipose tissues. Biochem Biophys Res Commun 2005; 332(2):392-7.
15. Kang H W, Lee S G, Otieno D, Ha K. Flavonoids, potential bioactive compounds, and non-shivering thermogenesis. Nutrients. 2018; 10: 9.
16. Azhar Y, Parmar A, Miller C N, Samuels J S, Rayalam S. Phytochemicals as novel agents for the induction of browning in white adipose tissue. Nutr Metab (Lond.) 2016; 13:89.
17. Lee E, Surh Y J. Induction of apoptosis in HL-60 cells by pungent vanilloids, [6]-gingerol and [6]-paradol. Cancer Lett 1998; 134:163-168.
18. Umukoro S, Ashorobi R B. Further studies on the antinociceptive action of aqueous seed extract of Aframomum melegueta. J Ethnopharmacol 2007; 109:501e504.
19. Umukoro S, Ashorobi R B. Further pharmacological studies on aqueous seed extract of Aframomum melegueta in rats. J Ethnopharmacol 2008; 115:489e493.
20. Wei C K, Tsai Y H, Korinek M, Hung P H, El-Shazly M, Cheng Y B, et al., 6-paradol and 6-shogaol, the pungent compounds of ginger, promote glucose utilization in adipocytes and myotubes, and 6-paradol reduces blood glucose in high-fat diet-fed mice. Int J Mol Sci 2017: 18(1):168.
21. Iwami M, Mahmoud F A, Shiina T, Hirayama H, Shima T, Sugita J, et al., Extract of grains of paradise and its active principle 6-paradol trigger thermogenesis of brown adipose tissue in rats. Auton Neurosci 2011; 161(1-2):63-7.
22. Sugita J, Yoneshiro T, Hatano T, Aita S, Ikemoto T, Uchiwa H, et al., Grains of paradise (Aframomum melegueta) extract activates brown adipose tissue and increases whole-body energy expenditure in men. British J Nutr 2013; 110(4):733-8.

1. A method of promoting or maintaining a healthy body weight, comprising administering to a person in need thereof an effective amount of a composition comprising 6-paradol.

2. The method of claim 1, wherein said composition comprises between about 2% and about 15% 6-paradol.

3. The method of claim 1, wherein said composition further comprises at least one of 6-shogaol and 6-gingerol.

4. The method of claim 3, wherein said composition comprises about 8% 6-shogaol.

5. The method of claim 3, wherein said composition comprises about 17% 6-gingerol.

6. The method of claim 1, wherein said composition is an extract of Aframomum melegueta.

7. The method of claim 1, wherein said composition is an extract of Aframomum melegueta seeds.

8. The method of claim 1, wherein said person has a healthy body weight or is overweight.

9. The method of claim 1, wherein said composition is administered systemically.

10. The method of claim 1, wherein said composition is administered orally.

11. The method of claim 1, wherein said composition is in a form selected from the group consisting of a powder, liquid, pill, tablet, pellet, granule, capsule, soluble film, solution, spray, syrup, linctus, lozenge, pastille, chewing gum, chew, paste, vapor, suspension, emulsion, ointment, cream, lotion, foam, liniment, gel, drop, topical patch, buccal patch, bead, gummy, gel, sol, injection, and combinations thereof.

12. The method of claim 1, wherein said composition further comprises at least one of an excipient, pharmaceutical carrier, bulking agent, binding agent, filler, and diluent.

13. The method of claim 1, wherein said composition is administered at a dose of about 250 mg.

14. The method of claim 1, wherein said composition is administered two or more times per day.

15. The method of claim 1, wherein said composition further comprises at least one of a vitamin, mineral, amino acid, protein, carbohydrate, lipid, fatty acid, caffeine, flavoring, sweetener, and preservative.

16. The method of claim 1, wherein said composition is combined with a food, snack, nutritional supplement, dietary supplement, food supplement, or beverage.

17-43. (canceled)
